(19) United States
(12) Patent Application Publication
(10) Pub. No.: US 2005/0014181 A1
(43) Pub. Date: Jan. 20, 2005

(54) Methods and compositions for the assessment of polymer assembly

(76) Inventors: Zorina S. Galis, Indianapolis, IN (US); Chad E. Johnson, West Lafayette, IN (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/872,345

(22) Filed: Jun. 18, 2004

Related U.S. Application Data
(60) Provisional application No. 60/479,293, filed on Jun. 18, 2003.

(51) Int. Cl. C12Q 1/68; G01N 33/53; G01N 33/567; A61K 38/39

(52) U.S. Cl. 435/6; 435/7.2; 514/21

(57) ABSTRACT

Systems and methods for identifying modulators of cell-mediated polymer assembly are provided. One aspect provides a high throughput method for identifying modulators of supramolecular polymer assembly.

### CROSS REFERENCE TO RELATED APPLICATION

This application claims benefit of and priority to U.S. Provisional Patent Application No. 60/479,293 filed on Jun. 18, 2003.

### STATEMENT REGARDING FEDERALLY SPONSORED RESEARCH OR DEVELOPMENT

Aspects of the work described herein were supported in part by Grant Number EEC-9731643 awarded by the National Science Foundation, and Grant Number RO1-HL64689 awarded by the National Institutes for Health. Therefore, the U.S. government may have certain rights in the claimed subject matter.

### BACKGROUND

1. Technical Field

The disclosure is generally directed to methods for the assessment of polymer assembly, in particular for the assessment of extracellular supramolecular polymer assembly and the identification of agents that affect such processes.

2. Related Art

Among other important properties, the extracellular matrix provides a scaffolding structure to which cells are attached within tissues. Collagen is a major matrix component and the most abundant protein in the human body. Collagen's supramolecular organization imparts high mechanical strength to tissues.

Inborn or local imbalances in the normal synthesis, turnover, and formation of collagen fibrils lead to pathologic conditions with serious clinical consequences. For example, excessive fibrillar collagen accumulation has been found to be responsible for increased stiffness and constriction of tissues, including the decreased compliance of atherosclerotic arteries, and for formation of keloids and tissue fibrosis (Hill C., et al. (2001) Transforming growth factor-beta2 antibody attenuates fibrosis in the experimental diabetic rat kidney. J Endocrinol. 170: 647-651). On the other hand, poor mechanical properties of arteries have been linked to deficient collagen fibrils in Ehlers-Danlos syndrome (Michalickova, K., et al. (1998) Mutations of the alpha2(V) chain of type V collagen impair matrix assembly and produce Ehlers-Danlos syndrome type I. Hum Mol Genet. 7:249-255) and abdominal aortic aneurysm (Bode, M. K., et al. (2000) Increased amount of type III pN-collagen in human abdominal aortic aneurysms: Evidence for impaired type III collagen fibrillogenesis. J Vasc Surg. 32:1201-1207).

Appropriate mechanical strength also is essential for tissue-engineered constructs, especially for those intended to resist significant mechanical stresses upon implantation into the body, such as tissue-engineered arterial conduits. For such applications it is especially important to be able to test and monitor the effect of various conditions upon the appropriate formation of higher-order structures of collagen.

While the process of collagen synthesis by cells has been intensively investigated and elucidated, less is known with regard to the contribution of cells to the post-translational assembly of collagen and the formation of fibrils, a process known as fibrillogenesis. To date, collagen fibril formation in vitro has been investigated mostly in the absence of cells (Brightman, A. O. (2000) Time-lapse confocal reflection microscopy of collagen fibrillogenesis and extracellular matrix assembly in vitro. Biopolymers 54:222-234; Cabral, W. A., et al. (2002) Procollagen with skipping of alpha 1(I) exon 41 has lower binding affinity for alpha 1(I) C-telopeptide, impaired in vitro fibrillogenesis, and altered fibril morphology. J Biol Chem 277:4215-4222; Perret, S. (2001) Unhydroxylated triple helical collagen I produced in transgenic plants provides new clues on the role of hydroxyproline in collagen folding and fibril formation. J Biol Chem 276:43693-43698). These studies have indicated that self-assembly of collagen molecules, which have low solubility at neutral pH, occurs at concentrations above physiologic levels. Alternatively, cell-mediated assembly of newly formed collagen molecules into fibrils has been investigated in cell-seeded collagen gels after fixation and histological processing (Kypreos, K. E., et al. (2000) Type V collagen regulates the assembly of collagen fibrils in cultures of bovine vascular smooth muscle cells. J Cell Biochem 80:146-155).

Currently technology for investigating extracellular matrix assembly does not provide a means for investigating cell-mediated extracellular formation of molecules in real-time. Accordingly, there is a need for new systems and methods of investigating cell-mediated polymer assembly.

### SUMMARY

Aspects of the present disclosure generally provide systems and methods for monitoring, detecting, and quantitatively assessing of the ability of live cultured cells to organize both endogenous and exogenous polymers, including, put not limited to collagen, under various in vitro conditions.

Another aspect provides a method for identifying compounds that modulate the assembly of polymers, for example supramolecular complexes including, but not limited to polypeptide complexes such as collagen and thrombin. Supramolecular assembly of biological polymers includes extracellular matrix or other extracellular biological material. Monomers can be individual units of a polymer or polymers themselves when for example at least two polymers combine to form a supramolecular structure, for example collagen. Monitoring matrix assembly can be accomplished using fluorescence microscopy in real-time on live cells or on cells fixed at different time periods.

Another aspect provides a system and method for identifying modulators of cell-mediated polymer assembly, particularly extracellular polymer assembly which is automated or configured for high throughput screening assays. One such high throughput assay can screen for modulators of cell-mediated polymer assembly using fluorescence imaging devices.

### BRIEF DESCRIPTION OF THE DRAWINGS

FIGS. 1A-C are fluorescence micrographs showing cell-mediated assembly of polypeptides,

FIGS. 1D-F are transmission electron micrographs of smooth muscle cells showing collagen assembly.

FIG. 2A is a bar graph showing that one embodiment of the discloses assay can distinguish between the cell-mediated assembly of labeled polypeptides.

FIG. 2B is a gel showing that collagen is assembled by living cells.

FIGS. 3A-B are line graphs showing the effect of increasing monomer concentration on cell-mediated collagen assembly.

FIGS. 3C-D are fluorescence micrographs showing the quantification of fibrillogenesis using a fluorescence plate reader.

FIGS. 4A-B are line graphs showing the effects of different sources of collagen on fibrillogenesis.

FIGS. 5A-C are line graphs showing the effect of various substances on cell-mediated polymer assembly using one embodiment of the disclosed assay.

### DETAILED DESCRIPTION

While the making and using of various embodiments of the present disclsoure are discussed herein in terms cell-mediated polymer assembly, it should be appreciated that the present disclosure provides many applicable inventive concepts that can be embodied in a wide variety of specific contexts. The specific embodiments discussed herein are merely illustrative and are not meant to limit the scope of the disclosure in any manner.

1. Definitions

The following definitions are helpful in understanding the present invention:

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); bis-BTC; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FI; Bodipy FL ATP; Bodipy FI-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP—Cyan Fluorescent Protein; CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5 ™; Cy3™; Cy5.1 8; Cy5.5™; Cy5 ™; Cy7 ™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′ DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiIC18(5)); DIDS; Dihydorhodamine 123 (DHR); DiI (DiIC18(3)); Dinitrophenol; DiO (DiOC18(3)); DiR; DiR (DiIC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP(S65T); GFP red shifted (rsGFP); GFP wild type, non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1, low calcium; Indodicarbocyanine (DiD); Indotricarbocyanine (DiR); Intrawhite Cf; JC-1; JO-JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; PhotOResist; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PYMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3 or a combination thereof.

The term “stem cell” means an undifferentiated cell or uncommitted cell. Such cells are also known in the art as totipotent cells and have the ability to produce any type of cell in the organism. Stem cells include embryonic stem cells, adult stem cells, bone marrow stem cells, or any other cell that can produce at least two different cell types for example mesoderm, ectoderm, or endoderm.

The term “pluripotent cell” means a cell that can produce at least two different cell types, typically two different cell types within a single type of tissue, for example mesoderm, ectoderm, or endoderm.

2. Methods and Assays

One of the several embodiments provides a system and method for identifying modulators of cell-mediated polymer assembly, in particular supramolecular assembly including but not limited to collagen fibrillogenesis. The cell-mediated polymer can be endogenous polymers or exogenous polymers. The polymers assembled by the cells can be formed of endogenous monomers, exogenous monomers, or a combination thereof.

Supramolecular complexes refers to a three dimensional assembly of more than one polymeric subunit, for example collagen fibrillogenesis. An exemplary supramolecular complex can be formed by the aggregation a plurality of linear arrays of monomers. The supramolecular complex can be formed by the assembly of more than one identical subunits or by more than one type of subunits. For example, fibrils of collagen are generally composed of molecules of tropocollagen in linear arrays. In Type I collagen, the most common type, the tropocollagen molecules are associated in periodic, staggered arrays that give the appearance of cross-banding, with a period of approximately 65 nm in the unit fibril (or microfibril); these unit fibrils are aggregated in bundles to form larger fibrils, with longitudinal striations, which may themselves be aggregated into fibers. Some other types of collagen also associate into fibrils (e.g., Types II, III, VI) but may not aggregate to show cross-banding or to form fibers. The terms fiber and fibril are sometimes interchanged loosely in descriptions of the hierarchy of collagen aggregation.

Generally, cells, for example smooth muscle cells or any other cell capable of producing and secreting monomers of a polymer, for example collagen monomers, or any cell capable of mediating extracellular polymer assembly, are seeded into the wells of a sample plate and maintained under standard cell culture conditions for a period of time, for example 24 h. It will be appreciated that any size or dimension of sample plate can be used. Each well or plate can optionally include a three-dimensional scaffold. After 24 h, the media is optionally replaced with serum free media for a period of time sufficient to promote cell quiescence, typically about 24 h.

A compound or series of compounds suspected of modulating cell-mediated polymer assembly such as collagen fibrillogenesis, are added to the cells. The test compound can be used to pretreat the cells, or the test compounds can be delivered to the cells concurrently with labeled monomers.

The compounds can be delivered to the cells using a fluid delivery device, for example an automated fluid delivery device. The fluid delivery device can be one or more motorized pipettes, for example micropipettes, configured to deliver the test compound to one or more wells of the sample plate. Volumes ranging from about 1 to about 100 μl are typically delivered to each well. The pipettes can be attached to a drive system so they can be lowered and raised relative to the plate to deliver fluid to each well.

The plate containing the test compounds can then be incubated for a period of time sufficient to permit the compound to exert an effect on the cells in the plate. The cells can be incubated under conditions that promote cell-mediated polymer formation. Such conditions include approximate physiological conditions for temperature, humidity, and pH, as well as concentrations of monomers or polymeric subunits sufficient to permit polymer assembly. Representative incubation periods can be as small as seconds to as long as days depending on the compound to be tested. Typically, 0.5 to 24 h incubation periods have been found to be sufficient. The concentration of the test compound can be delivered in ranges to determine the optimal dose for the test compound. The test compound can be maintained in the well throughout the assay or can be removed prior to the addition of the labeled monomers.

Labeled monomers are added to the wells of the sample plate. Different types of monomer subunits can be labeled with different types of detectable markers or each monomer can be labeled with the same label. Suitable monomer concentrations are about 10-100 μg/L, but can vary depending on the monomer and compound to be tested. The label can be any detectable label. Detectable labels and methods of attaching them to biomolecules are known in the art (See Molecular Probes Handbook at www.probes.com which is incorporated by reference in its entirety). Preferred labels include, but are not limited to, optically detectable labels such as chromophores and fluorophores. It will be appreciated that any detectable fluorophore that can be linked to the monomer can be used in the disclosed system and methods.

Once the labeled monomers are added to the sample plate, the cells are incubated under standard cell culture conditions for a period of time sufficient to promote cell-mediated polymer assembly. Time periods ranging from about 1 to about 24 h were observed to be sufficient. After which, the cells are washed and the media replaced with phosphate buffered saline. In one embodiment, a fluorescent label is used and fluorescence from each well is measured using a plate reader or visualized using fluorescent microscopy. Detectable fluorescence in samples with the test compound can be compared to control samples. Control samples do not contain the test compound but typically contain all the other components of the assay. The cell-mediated polymers can include labeled monomers as well as unlabeled monomers or can consist entirely of labeled monomers.

Compounds that modulate the detectable signal of a test sample compared to the detectable signal of the control sample are identified as modulators of cell-mediated polymer assembly. Such compounds can promote or inhibit cell-mediated polymer assembly.

Another embodiment provides a method for identifying modulators of cell-mediated extracellular matrix assembly by contacting a cell with a test compound, contacting the cell with labeled monomers of an extracellular matrix polymer, incubating the cell in the presence of the labeled monomers under conditions that promote extracellular matrix assembly, optionally washing the cell to remove unpolymerized labeled monomers, and detecting remaining labeled monomers. The amount of detectable remaining labeled monomers can be correlated with extracellular matrix assembly. An increase in detectable signal in cells treated with the test compound compared to cells that were not treated with the test compound indicates that the test compound promotes extracellular matrix assembly. A decrease in detectable signal in cells treated with the test compound compared to cells that were not treated with the test compound indicates that the test compound inhibits extracellular matrix assembly. As noted above, the cell can be seeded into a sample plate having a scaffold. Accordingly, the disclosed systems and methods include identifying compounds or agents that affect the assembly of a three-dimensional extracellular complex which includes but is not limited to, the extracellular matrix or a component thereof.

The systems and methods of the disclosure can be used to identify agents for the treatment of a pathology related to or caused by dysfunctional cell-mediated polymer assembly. Exemplary diseases include, but are not limited to, Ehlers-Danlos Syndrome, osteogenesis imperfecta, and Marfan syndrome.

Alternatively, the disclosed systems and methods can be used to identify agents that improve or facilitate the generation of tissues and tissue scaffolds in vitro, including but not limited to arterial conduits, valves, cartilage, and the like, or tissue engineered or biodegradable scaffolds. Such agents can be used in combination with tissue engineering to rapidly generate specific tissues using extracellular matrix as a scaffold. Agents that direct the formation of the extracellular matrix into specific structures, geometries, or configurations can also be identified.

2.1 Types of Cells

Embodiments of the disclosure provide systems and assays including living cells. Generally, animal or plant cells that produce a monomeric or polymeric subunit of a polymer or supramolecular complex can be used. Representative cells include mammalian cells such as human cells. The cells can be primary culture cells, immortablized cells, transfected cells, stem cells, pluripotent cells, umbilical blood cells, bone marrow cells, embryonic stem cells, adult stem cells, smooth muscle cells, or the like. Transfected cells includes those cells that express an exogenous nucleic acid, typically an exogenous nucleic acid that encodes a monomer of a polymer, for example, a collagen polypeptide. Techniques for transfecting cells are known in the art.

Additionally, it will be appreciated that cells may be transfected or otherwise manipulated to express altered genes or proteins believed to be involved in cell-mediated polymer assembly, in particular, extracellular matrix assembly. The effect of these altered genes or proteins can be assessed using the disclosed systems and methods. Representative genes and proteins that can be altered include, but are not limited to, collagen, fibronectin, thrombin, signal transduction proteins, cell surface proteins, receptor proteins, actin, tublin, transcription factors, and the like.

2.2 Polymers

Cell-mediated assembly of polymers generally refers to the extracellular assembly of natural polymers by living cells. The polymers can be assembled into supramolecular structures such as collagen fibrils. Natural polymers include, but are not limited to polypeptides, polynucleotides, carbohydrates, lipids, glycosaminoglycans, polysaccharides, proteoglycans, and fibronectin. Exemplary glycosaminoglycans include but are not limited to hyaluronate, chondroitin sulfate, heparan sulfate, heparin, dermatan sulfate, and keratan sulfate.

A representative polymer and supramolecular complex is collagen. The formation of collagen begins with the transcription of α-chains. Three α-chains combine to form procollagen. Procollagen is secreted into the extracellular space where it is cleaved by procollagen peptidases to form collagen. Collagen then is assembled into fibrils. Multiple collagen units combine to form one fibril. This assembly is mediated by cells and is driven to some extent by self-assembly. Another supramolecular complex is formed by thrombin, for example during the formation of a clot or thrombosis.

2.3 Detectable Labels

It will be appreciated that any detectable label can be used in the disclosed systems and methods to detect and monitor cell-mediated polymer assembly. Suitable labels include, but are not limited to biotin, chromophores, fluorophores, metal particles having a diameter of less than about 10 nm, magnetic particles, enzymes, radioisotopes, isotopes, spin labels, colloidal gold or silver, antibodies or fragments thereof, and the like. Some embodiments of the disclosed methods and systems include the use of more than one type of label. For example, some monomers or polymeric subunits can be labeled with a fluorophore that emits at a first wavelength, and other monomers can be labeled with a second fluorophore that emits at a second wavelength. In one embodiment, the labels are selected in pairs so that Fluorescence Resonance Energy Transfer (FRET) occurs between the two fluorophores when the two fluorophores come within close proximity to one another, typically within about 100 Å. In this embodiment, a detectable signal at a specific wavelength is generated only when polymers or supramolecular complexes are assembled.

In other embodiments, the absorption spectrum of the second label or quencher overlaps with the emission spectrum of the first label or fluorophore. Many donor/quencher dye pairs known in the art are useful in some embodiments of the present disclosure. These include, for example, fluorescein isothiocyanate (FITC)/tetramethylrhodamine isothiocyanate (TRITC), FITC/Texas Red™ (Molecular Probes), FITC/N-hydroxysuccinimidyl 1-pyrenebutyrate (PYB), FITC/eosin isothiocyanate (EITC), N-hydroxysuccinimidyl 1-pyrenesulfonate (PYS)/FITC, FITC/Rhodamine X, FITC/tetramethylrhodamine (TAMRA), and others. P-(dimethyl aminophenylazo) benzoic acid (DABCYL) is a non-fluorescent quencher dye which effectively quenches fluorescence from an adjacent fluorophore, e.g., fluorescein or 5-(2′-aminoethyl) aminonaphthalene (EDANS). In this embodiment, a signal is detectable in the absence of polymer assembly.

Methods for linking labels to biomolecules are known in the art. Of the various linking chemistries that can be used to link molecules with other molecules or reagents, the most common are amine, carbonyl, carboxyl, and thiol. It will be appreciated by those of skill in the art, that any linking chemistry may be utilized. Indirect crosslinking of the amines in one molecule to the thiols in a second molecule is the predominant method for forming a heteroconjugate. If the nucleic acid reporter, the spacer, or the protein transduction domain does not already contain one or more thiol groups, the thiol groups can be introduce using a thiolation procedure.

Thiol groups (also called mercaptans or sulfhydryls) are present in cysteine residues of proteins. Thiols can also be generated by selectively reducing cystine disulfides with reagents such as dithiothreitol (DTT) or -mercaptoethanol. Removal of DTT or -mercaptoethanol is sometimes accompanied by air oxidation of the thiols back to the disulfides. Reformation of the disulfide bond can be avoided by using the reducing agent tris-(2-carboxyethyl)phosphine (TCEP), which does not contain thiols. TCEP is generally impermeable to cell membranes and to the hydrophobic protein core, permitting its use for the selective reduction of disulfides that have aqueous exposure. The pH-insensitive and less polar phosphine derivative tris-(2-cyanoethyl)phosphine may yield greater reactivity with buried disulfides.

Several methods are available for introducing thiols into molecules, including the reduction of intrinsic disulfides, as well as the conversion of amine, aldehyde or carboxylic acid groups to thiol groups. Disulfide crosslinks, for example of cystines in proteins, can be reduced to cysteine residues by dithiothreitol, tris-(2-carboxyethyl)phosphine or tris-(2-cyanoethyl)phosphine.

Amines can be indirectly thiolated by reaction with succinimidyl 3-(2-pyridyidithio)propionate, followed by reduction of the 3-(2-pyridyldithio)propionyl conjugate with DTT or TCEP. Alternatively, amines can be indirectly thiolated by reaction with succinimidyl acetylthioacetate, followed by removal of the acetyl group with 50 mM hydroxylamine or hydrazine at near-neutral pH.

Thiols can also be incorporated at carboxylic acid groups by an EDAC-mediated reaction with cystamine, followed by reduction of the disulfide with DTT or TCEP. Tryptophan residues in thiol-free proteins can be oxidized to mercaptotryptophan residues, which can then be modified by iodoacetamides or maleimides.

Thiol-reactive functional groups are primarily alkylating reagents, including iodoacetamides, maleimides, benzylic halides and bromomethylketones. Arylating reagents such as NBD halides react with thiols or amines by a similar substitution of the aromatic halide. Reaction of any of these functional groups with thiols usually proceeds rapidly at or below room temperature in the physiological pH range (pH 6.5-8.0) to yield chemically stable thioethers.

Thiols also react with many of the amine-reactive reagents described in including isothiocyanates and succinimidyl esters. Although the thiol-isothiocyanate product (a dithiocarbamate) can react with an adjacent amine to yield a thiourea, the dithiocarbamate is more likely to react with water, consuming the reactive reagent without forming a covalent adduct.

Iodoacetamides readily react with all thiols, including those found in peptides, proteins and thiolated polynucleotides, to form thioethers. Iodoacetamides can sometimes react with methionine residues. They may also react with histidine or tyrosine, but generally only if free thiols are absent. Although iodoacetamides can react with the free base form of amines, most aliphatic amines, except the -amino group at a protein's N-terminus, are protonated and thus relatively unreactive below pH 8. In addition, iodoacetamides react with thiolated oligonucleotide primers, as well as with thiophosphates and thiouridine residues present in certain nucleic acids, but usually not with the common nucleotides.

Iodoacetamides are intrinsically unstable in light, especially in solution; reactions should therefore be carried out under subdued light. Adding cysteine, glutathione or mercaptosuccinic acid to the reaction mixture will quench the reaction of thiol-reactive probes, forming highly water-soluble adducts that are easily removed by dialysis or gel filtration. Although the thioether bond formed when an iodoacetamide reacts with a protein thiol is very stable, during amino acid hydrolysis the bioconjugate loses its fluorophore to yield S-carboxymethylcysteine.

Maleimides are excellent reagents for thiol-selective modification, quantitation and analysis. The reaction involves addition of the thiol across the double bond of the maleimide to yield a thioether. Maleimides apparently do not react with methionine, histidine or tyrosine. Reaction of maleimides with amines usually requires a higher pH than reaction of maleimides with thiols. Hydrolysis of maleimides to a mixture of isomeric nonreactive maleamic acids can compete significantly with thiol modification, particularly above pH 8. Furthermore, maleimide adducts can hydrolyze or they can ring-open by nucleophilic reaction with an adjacent amine to yield crosslinked products. This latter reaction can potentially be enhanced by raising the pH above 9 after conjugation.

For example, a disulfide-containing linker or spacer, including but not limited to an alkyl linker or spacer of about 1 to about 12 carbon atoms, is photo- or thermally coupled to the target nucleobase or polynucleotide using conventional chemistry, for example azide chemistry. The disulfide bond is reduced, yielding a free thiol. A covalent bond is formed between the reagent thiol and a thiol-reactive linker, hapten, fluorochrome, sugar, affinity ligand, or other molecule.

The linking of two molecules can be achieved using heterobifunctional crosslinkers. Representative heterobifunctional crosslinkers include, but are not limited to, p-maleimidophenyl isocyanate; succinimidyl acetylthioacetate; succinimidyl-trans-4(maleimidylmeythyl)-cyclohexane-1carboxylate (SMCC); succinimidyl acetylthioacetate (SATA); succinimidyl 3-(2-pyridyidithio)propionate (SPDP); N-((2-pyridyldithio)ethyl)-4-azidosalicylamide (PEAS; AET); 4-azido-2,3,5,6-tetrafluorobenzoic acid, succinimidyl ester (ATFB, SE); 4-azido-2,3,5,6-tetrafluorobenzoic acid, STP ester, sodium salt (ATFB, STP ester); 4-azido-2,3,5,6-tetrafluorobenzyl amine, hydrochloride; benzophenone-4-isothiocyanate; benzophenone-4-maleimide; 4-benzoylbenzoic acid, succinimidyl ester. The heterobifunctional crosslinkers can be photoreactive, amine and/or thiol reactive, or aldehyde/ketone reactive, or a combination thereof.

2.4 Scaffolds

The scaffold of the disclosed systems and methods may be composed of synthetic or natural materials including, but not limited to, proteins such as collagen, carbohydrates, hydrogel materials, synthetic materials, plastics, hydroxyapatite, tricalcium phosphate, foams, and combinations thereof. Representative synthetic materials include, but are not limited to, aliphatic polyesters such as polyglycolic acid (PGA) and polylactic acid (PLA), and their copolymers such as polycaprolactone, Polyglactin 910 (comprising a 9:1 ratio of glycolide per lactide unit, and known also as VICRYL™), polyglyconate (comprising a 9:1 ratio of glycolide per trimethylene carbonate unit, and known also as MAXON™), and polydioxanone (PDS). The polymers of the scaffold may be prepared by synthetic or natural methods. Generally, the scaffold polymer should not contain any undesirable residues or impurities which could elicit an undesirable response. Additionally, the scaffold should be biocompatible, and should not substantially interfere with optical imaging of cell-mediated polymer assembly. In one embodiment, the scaffold is composed of a translucent or transparent material.

Typical scaffolds are porous. One embodiment provides a scaffold that is a substantially homogeneous, solid structure, provided with small holes (pores), which enable diffusion of nutrients and waste products. Another embodiment provides a scaffold that is a fibrous structure, which is composed of different elements (fibers). The scaffold can be a continuous structure, substantially composed of one element, having distinct compartments. The pores can also be interconnected.

Preferably, the scaffold has a macroporosity between 30 and 99%, more preferably between 60 and 95%. The pores in the scaffold can have a diameter of between 0.1 and 2000 μm, typically between 1 and 1000 μm. The macroporosity and the diameter of the pores are chosen such that cell-mediated polymer assembly can be readily imaged using optical devices, including, but not limited to, fluorescence microscopy devices. Additionally, the diameter of the pores should be sufficient to permit supramolecular cell-mediated assembly of polymers.

In some embodiments, the scaffold can be formed of a specific class of polymeric materials having hydrogel properties. This is the class of copolymers of a polyalkylene glycol and an aromatic polyester. Preferably, these copolymers comprise 40-80 wt. %, more preferably 50-70 wt. % of the polyalkylene glycol, and 60-20 wt. %, more preferably 50-30 wt. % of the aromatic polyester. A preferred type of copolymers according to the invention is formed by the group of block copolymers.

The scaffold can be a composite material. Exemplary composite materials include ceramics in combination with biodegradable materials.

The scaffold material can be coated with a substance that promotes the adhesion of cells onto the scaffold. Such substances include, but are not limited to, poly-lysine or other positively charged polymers.

One embodiment includes a scaffold having one or more of the following properties: (1) interconnecting pores with dimensions that facilitate cell integration and nutrient exchange, (2) surface chemistry favoring cellular attachment, differentiation, and proliferation; (3) produces no adverse response by the cells; and (4) permits optical microscopy detection of cell-mediated polymer assembly.

The scaffolds can be prepared using techniques known in the art, for example, solid freeform fabrication including but not limited to solvent-casting particulate-leaching, gas foaming, fiber meshes, phase separation, melt molding, emulsion freeze drying, solution casting, and freeze drying (Sachlos, E. and J. T. Czernuszka (2003) Making Tissue Engineering Scaffolds Work. European Cells and Materials 5:29-40, which is incorporated by reference herein in its entirety).

2.5 High Throughput Assays

One embodiment provides a high throughput assay for identifying compounds that modulate cell-mediated polymer assembly including, but not limited to supramolecular complex assembly. Generally, the disclosed high throughput screens use mixtures of compounds and biological reagents along with some indicator compound loaded into arrays of wells in standard microtiter plates with 96 or 384 wells. The signal measured from each well, typically either fluorescence emission, optical density, or radioactivity, integrates the signal from all the material in the well giving an overall population average of all the molecules in the well.

A particular embodiment provides a high throughput assay system for identifying modulators of cell-mediated polymer assembly, in particular, extracellular cell-mediated polymer assembly and supramolecular polymer complexes. The system includes an optical system for detecting labeled cell-mediated polymers formed in the presence of absence of a test compound. An exemplary optical system includes, but is not limited to, a fluorescence optical system having a microscope objective, an XY stage adapted for holding a plate with an array of locations for holding cells and having a means for moving the plate to align the locations with the microscope objective and a means for moving the plate in the direction to effect focusing; a digital camera; a light source having optical means for directing excitation light to the array and a means for directing fluorescent light emitted from the plate to the digital camera; and a computer means for receiving and processing digital data from the digital camera. The computer means can include: a digital frame grabber for receiving the images from the camera, a display for user interaction and display of assay results, digital storage media for data storage and archiving, and means for control, acquisition, processing and display of results.

Imaging plate readers are known in the art. A typical system uses a CCD camera to image the whole area of a 96 well plate. The image is analyzed to calculate the total fluorescence per well for all the material in the well. Proffift et. al. (Cytometry 24: 204-213 (1996)) describe a semi-automated fluorescence digital imaging system for quantifying relative cell numbers in situ in a variety of tissue culture plate formats, especially 96-well microtiter plates. The system consists of an epifluorescence inverted microscope with a motorized stage, video camera, image intensifier, and a microcomputer with a PC-Vision digitizer. Turbo Pascal software controls the stage and scans the plate taking multiple images per well. The software calculates total fluorescence per well, provides for daily calibration, and configures easily for a variety of tissue culture plate formats. Thresholding of digital images and reagents which fluoresce only when taken up by living cells are used to reduce background fluorescence without removing excess fluorescent reagent.

Molecular Devices, Inc. (Sunnyvale, Calif.) describes a system (FLIPR) which uses low angle laser scanning illumination and a mask to selectively excite fluorescence within approximately 200 microns of the bottoms of the wells in standard 96 well plates in order to reduce background when imaging cell monolayers. This system uses a CCD camera to image the whole area of the plate bottom. Although this system measures signals originating from a cell monolayer at the bottom of the well, the signal measured is averaged over the area of the well and is therefore still considered a measurement of the average response of a population of cells. The image is analyzed to calculate the total fluorescence per well for cell-based assays. Fluid delivery devices have also been incorporated into cell based screening systems, such as the FLIPR system, in order to initiate a response, which is then observed as a whole well population average response using a macro-imaging system.

Additional optical imaging techniques include but are not limited to reflection microscopy, autofluorescence microscopy, confocal microscopy, and multiphoton microscopy (Voytik-Harbin, S. L. et al., (2001) Three-dimensional imaging of extracellular matrix and extracellular matrix-cell interactions. Methods Cell Bio 63:583-97, which is incorporated by reference in its entirety).

3. Kits

Another embodiment provides a kit for monitoring, detecting, or quantifying cell-mediated polymer assembly. Representative kits include a sample plate for culturing cells optionally including a scaffold. The sample plate is typically configured for optical detection of labeled polymers. Labeled monomers or polymeric subunits can also be included. Representative labeled monomers or polymeric subunits include, but are not limited to polypeptides such as collagen. Instructions for using the kit can also be included as well as appropriate buffers and reagents.

### EXAMPLES

Reagents

Acid soluble rat-tail collagen (3.4-4.0 mg/mL) and bovine-skin collagen (3.5 mg/mL) (Becton Dickinson) in 0.02M of acetic acid and bovine serum albumin (Sigma) were stored at 4° C. until use. Ascorbic acid (Sigma) from powder was prepared immediately before use in the culture media. Retinoic acid (Sigma), prepared at 167 mM in DMSO, and the FLUOS labeling kit (Roche) were kept at 4° C. and protected from light until use. Cytochalasin D (Sigma) was prepared at 1 mM in chloroform and stored at −20° C. until use. DMEM, L-glutamine, penicillin, streptomycin, and 0.25% trypsin in EDTA were purchased from Cellgro. Fetal bovine serum (FBS, Sigma) was used as a cell growth supplement. Nuclear stain Hoechst 33258 (Sigma) was prepared at 500 g/mL in methanol and stored at −20° C. protected from light.

### Example 1

### Vascular Smooth Muscle Cell Isolation and Culture

Mouse aortic smooth muscle cells (SMC) were isolated from explants of aortas harvested from mice in the 129 SvEv genetic background. Briefly, the aortas were excised, the adventitia was removed, the aorta cut into approximately 1-mm rings, and the rings plated on scored areas of a Petri dish. After 2 weeks, the aortic rings were removed and the cells were grown for an additional 2 weeks before passaging. The primary culture and the first passage of SMC were grown in DMEM supplemented with 20% FBS, 1% L-glutamine and penicillin-streptomycin, followed by DMEM with 10% FBS for subsequent cultures.

An explant method also was used to isolate SMC from human saphenous vein. Human saphenous vein segments, obtained as excess after coronary bypass surgery, were cut longitudinally, unfolded, and adventitia and intima were removed using blunt dissection with forceps. The media was minced and plated onto Petri dishes, as described above, for aortic rings. Human SMC could be seen migrating out of tissue segments at 1 week after initial plating. All cell culture conditions were the same as for mouse aortic SMC. All SMC used in these experiments were from passages 3 to 8. Identity of the cells was confirmed by immunocytochemical detection of -smooth-muscle-cell actin. Purity was >95% for all SMC cultures.

### Example 2

### Protein Labeling

To verify the ability of the assay to discriminate cell-mediated protein assembly, collagen monomers were compared with BSA and denatured collagen. A bovine serum albumin (BSA) solution (4 mg/mL in deionized water) and acid solubilized rat-tail or bovine-skin collagen were dialyzed separately against borate-buffered saline (100 mM of sodium borate, 1 M of sodium chloride, pH 9.3) using dialysis tubing (8000 MWCO, Spectrum Medical Industries), for 24 h at 4° C.

At this pH, necessary for the fluorescein labeling, the concentrated collagen solution gelled. The dialyzed protein solutions were transferred to reaction vessels to which 7.9 L of FLUOS [Roche, 20 mg/mL fluorescein isothiocyanate (FITC) in DMSO] per mg of protein were added (an approximately 56:1 molar ratio of FLUOS to protein). The labeling mixture was allowed to react for 3 h at room temperature with gentle stirring and protected from light.

After labeling, the FLUOS-protein mixture was transferred into dialysis tubing and dialyzed against 0.02M of acetic acid for 24 h at 4° C. to remove any unbound FITC and to resolubilize the labeled collagen. Throughout the procedure, the labeled protein was protected from light. The dialyzed FITC-labeled protein solutions then were collected and stored at 4° C.

For further testing of the assay, FITC-labeled rat-tail collagen was heated to 95° C. for 15 min to denature the collagen in order to assess assembly characteristics. Final protein concentrations were determined using a DC protein assay (Biorad). Labeling efficiency (FITC molecules/protein molecules) was determined by comparison to a standard curve raised using known amounts of the FLUOS labeling stock and normalized by the molarity of the protein.

Under the conditions used, labeling efficiency varied from 3.0 to 5.3 FITC molecules per collagen molecule across all the batches, with an average of 4.6+0.5 FITC labels per collagen molecule. As the initial molar ratios of FITC-to-collagen molecule were approximately 56:1, the overall labeling efficiency was approximately 8.2%. Bovine serum albumin was labeled at a comparable level of 4.3 FITC molecules per BSA molecule. Gelling properties of the FITC-labeled collagen were tested using changes in optical density (=340 nm) for the control (non-neutralized) and neutralized collagen at room temperature and 37° C. In all conditions, the FITC-labeled collagen had no impairment in gelation, as evidenced by statistically identical changes in optical density.

### Example 3

### Protein Assembly Assay

In one embodiment, the assay consists of adding a solution of FITC-labeled protein to cultured cells in the presence or absence of various treatments, then returning the cells to the incubator (37° C., 5% CO₂) for various periods of time. Supramolecular protein assembly can be followed in time as a gain of fluorescence using a fluorescence plate reader, or by confocal microscopy with live cells or after fixation of cells. Specifically, for the development of this assay, SMC were used that had been plated into black clear-bottomed 96-well tissue culture-treated plates (Fisher) at 10,000 cells per well in 100 L of DMEM+10% FBS 2 days prior to performing the assay.

After 24 h, the culture medium was changed to DMEM without FBS to promote cell quiescence, then SMC were returned to the incubator (37° C., 5% CO₂) for an additional 24 h. Before the start of the assay, the culture medium was aspirated from each well and replaced with 100 L of FITC-labeled collagen, BSA, or denatured collagen in DMEM without FBS, final concentration between 10-100 g/mL. The cells were returned to the incubator for time periods up to 24 h.

At the end of the assay, the culture medium was aspirated and replaced with 10% buffered formalin (Fisher) for 10 min. For normalization of results, cell nuclei were counterstained using HOECHST [500 ng/mL in phosphate-buffered saline (PBS), pH 7.4] for 5 min. The wells subsequently were washed two times with PBS, then 100 L of PBS were added to each well and the intensity of the fluorescence was measured with a fluorescence plate reader (CytoFluor® series 4000, PerSeptive Biosystems) using the following settings for protein assembly: ex=480 nm, em=530 nm, gain 85, and ex=360 nm, em=460 nm, gain 80 for cell nuclei.

In each quantitative assay, twelve wells of a 96-well plate were used for each condition. Average background readings were obtained from wells without cells or without labeled collagen monomer solution (six each) to ensure that the assay measured only the cell-assisted protein assembly. Protein assembly for each condition was measured as the average intensity of green fluorescence above the background readings from wells that did not contain cells, normalized by the cell density, and determined as the intensity of blue fluorescence after subtracting the background readings from cell-free wells.

For visualization of the process by fluorescent microscopy, SMC were plated in 8-well tissue culture chamber slides (Labtek) at 20,000 cells per well and treated as above. After the final PBS wash, the liquid was removed and the slides were cover-slipped using Fluormount (Dako). Images were acquired with a Zeiss Axioscope™ using a Photonics camera connected to a computer.

### Example 4

### Assessment of the Novel Protein Assembly Assay

To establish the assay and test its capabilities, the labeled monomers of collagen, a protein known to be assembled by cells in vivo were used. The collagen assembly assay was performed under multiple conditions (n=12 wells for each condition). For the time course analysis, the assay was performed after 0, 1, 2, 4, 6, 8, 16, and 24 h, staggering the beginning times by starting with the wells used for the data longer time points first in order to measure all samples simultaneously at the end of the experiment.

To test the effect of collagen monomer concentration upon the assay, assembly was measured at 24 h after addition of FITC-labeled collagen monomers at 0, 10, 30, 50, 75, and 100 g/mL of final concentrations. The collagen monomer solution did not form fibrils spontaneously (in the absence of cells) at these concentrations. Above 100 g/mL, the collagen solution tended to spontaneously form loose collagen gels, obscuring cell-collagen interaction. In order to determine if the assay works with collagen and with cells from different sources, the assay was repeated using labeled monomers of rat-tail collagen, in the same range of concentrations, and SMC derived from human saphenous vein.

To assess the ability of our assay to detect the effects of treatments that interfere with cell cytoskeleton, and therefore may affect fibrillogenesis, SMC were pretreated with 1, 5, and 10 M of cytochalasin D, a chemical that disrupts organization of the actin cytoskeleton, for 30 min prior to the assay. The same concentrations were maintained throughout the assay. Retinoic acid, which has the opposite effect, stiffening SMC cytoskeleton, was added at 1, 5, and 10 M of final concentrations.

To test whether the assay can detect formation of fibrils by incorporating de novo synthesized endogenous collagen, effect of treatments that should directly affect this process were investigated. Ascorbic acid, which enhances the triple helix organization of de novo synthesized collagen, was added at 5, 10, and 50 M of final concentrations to the SMC culture medium for 24 h prior to the assay and maintained throughout the assay. Homocysteine thiolactone, which is a lysyl oxidase inhibitor and thus impairs the higher organization of collagen, was used at 5, 10, 50, 100, and 500 M of final concentration in the SMC culture media. To assess the potential effects of these treatments, fibrillogenesis was calculated as the percentage of fibrillogenesis mediated by nontreated SMC.

### Example 5

### Test for Specificity of Assay for FITC-Labeled Protein Assembly by SMC

SMC were plated in 6-well tissue culture plates at a density of 500,000 cells per well. After 24 h, the media was aspirated and replaced with 1 mL of serum-free media containing 50 or 100 g/mL of FITC-labeled BSA, collagen, or denatured collagen and placed back in a 37° C., 5% CO₂ incubator for 24 h for assembly. The SMC cultured with labeled proteins then were rinsed twice with PBS, after which cell extracts were taken using 100 L of acidified (0.02M of acetic acid) lysis buffer. Samples (20 L) from each condition before and after culturing with SMC were run on an 1.5-mm SDS-PAGE gel (Biorad apparatus) for 2 h at 150 V. FITC-labeled protein bands were imaged using a phosphoimager (Storm 860, Molecular Dynamics) with the ability to detect green fluorescence.

### Example 6

### Specificity of Assay for Proteins Known to Form Supramolecular Complexes

To verify that the assay was specific for supramolecular organization of collagen, the results obtained using FITC-labeled collagen monomers with or without previous heat denaturation, as heat denaturation of collagen should prevent formation of collagen fibrils were compared. Bovine-serum albumin (BSA) was used to determine if the increase in fluorescence intensity was due to nonspecific binding or to the uptake of protein. It was found that the assay was specific for the assembly of nondenatured collagen.

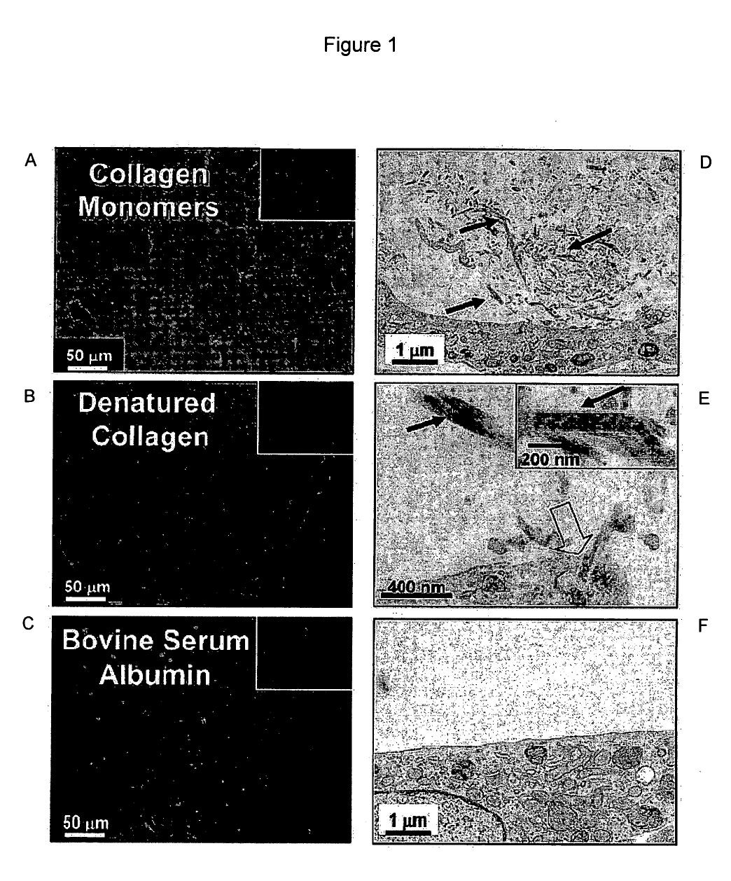

Labeled collagen incubated with SMC for 24 h formed the classical striated pattern of collagen fibrils (FIG. 1), but although labeled denatured collagen had some accumulation at the cell surface, it did not demonstrate assembly. No assembly was detected in cultures incubated with labeled BSA. Using the fluorescent plate reader to quantify increase in signal, it was found that similar results with collagen monomers displaying nearly fivefold higher values than BSA or denatured collagen (p<0.01 for collagen vs. BSA or denatured collagen at either concentration). These results demonstrate that this assay is able to specifically measure collagen assembly.

FIG. 1 shows the visual assessment of an exemplary embodiment of the assay, which can differentiate between the cell-mediated assembling of fluorescently labeled proteins. In FIGS. 1A-C (fluorescence microscopy), an exemplary assay was performed adding FITC-labeled proteins, that is, bovine serum albumin, collagen, and denatured collagen (50 and 100 mg/mL) to live cells. The only higher-ordered structures indicated by the green fluorescent label were obtained with nondenatured collagen monomers. Heat-denatured collagen had a defective assembly, and BSA formed only small aggregates. Hoechst nuclear staining (blue fluorescence) was used to measure cell density (insets).

FIGS. 1D-F are transmission electron microscopy (TEM) images of SMC incubated with 50 g/mL of collagen or with no collagen (control) for 24 h. FIG. 1D is a TEM image of FITC-labeled collagen assembly by SMC after 24 h. Arrows point to collagen assembled into fibrils. Middle: Higher magnification TEM illustrating a collagen fibril extending out of the SMC surface (block arrow), suggesting the role of cell surface crypts in collagen localization and assembly. Inset: Note the classical striated appearance of collagen fibrils (arrow). FIG. 1F is a TEM image of SMC that were kept in culture for 24 h without exogenous collagen (control). Note the apparent lack of extracellular matrix, specifically collagen fibrils.

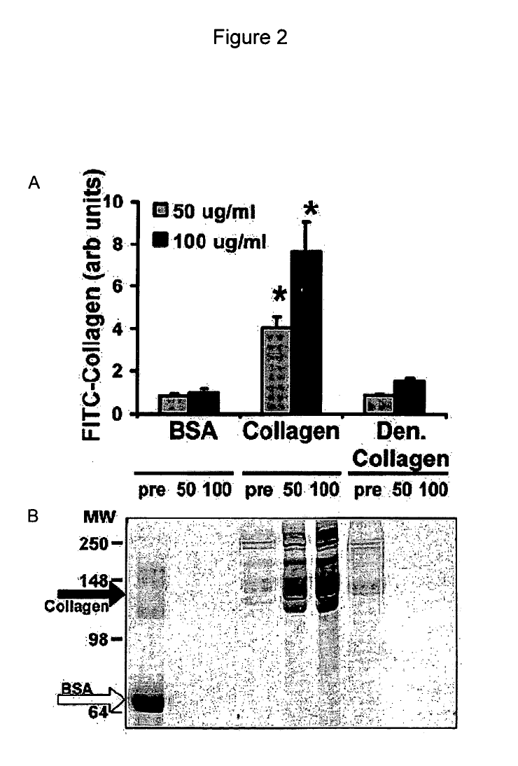

To investigate whether or not the collagen remained intact after the interaction with live cells, SDS-PAGE was performed on both the initial labeled and resolubilized collagen samples after incubation with SMC. As shown in FIG. 2, initially FITC-labeled BSA, collagen, and denatured collagen appear clearly and at approximately appropriate molecular weights (65 kD for BSA and 100 kD for collagen and denatured collagen, with some multimers present). FIG. 2 shows the quantitative and biochemical assessment of polymer assembly. In this embodiment the exemplary assay can differentiate between the cell-mediated assembling of fluorescently labeled proteins. The assay was performed by adding 50 and 100 mg/mL of FITC-labeled bovine serum albumin, collagen, or denatured collagen to SMC. Top graph: The quantification performed using a fluorescent plate reader indicated a significant (*p<0.05 for collagen vs. BSA or denatured collagen at same concentration) increase in green fluorescence normalized by cell density (blue fluorescence).

FIG. 2B shows the fluorescence detection of proteins after SDS-PAGE using a phosphoimager. Samples were fluorescently labeled proteins loaded before (pre) or collected after incubation (at 50 or 100 mg/mL) with SMC in culture for 24 h. The FITC-labeled collagen was recovered from the SMC monolayer, suggesting its association and assembly by cells, whereas neither BSA nor denatured collagen was associated with cells at either concentration.

Fluorescence levels of collagen and denatured collagen were similar, indicating no detrimental effect of heat denaturing on the FITC label. After incubation with SMC, only strongly fluorescent collagen bands were detected, indicating that assembly was necessary for accumulation of labeled protein. The SDS-PAGE gel further showed that there was no significant buildup of degraded collagen products, illustrating that the assay is specific for intact collagen assembly.

### Example 7

### SMC-Mediated Collagen Fibrillogenesis is Time- and Concentration-Dependent

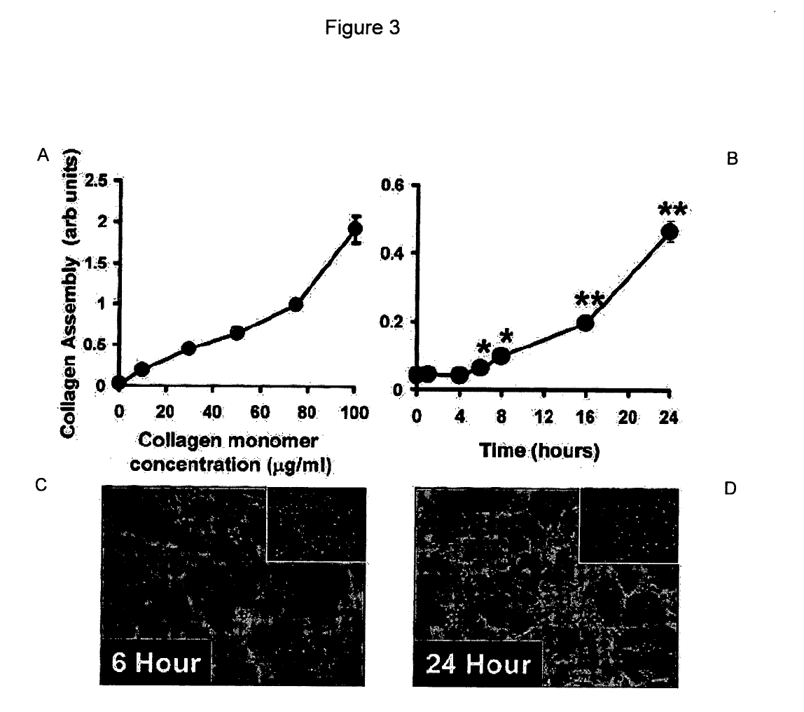

To test whether the assay could measure the dependence of assembly concentration upon collagen assembly, the fluorescence associated with SMC-dependent fibrillogenesis at collagen concentrations between 0-100 g/mL were investigated (FIG. 3). Using fluorescence microscopy, and through measurement, using the fluorescence plate reader, the highest concentration used in this assay for collagen monomers (100 g/mL) did not result in spontaneous formation of fibrils (i.e., fluorescence intensity at the highest concentration was still under the detection level).

FIG. 3 shows the effect of collagen monomer concentration and time course upon SMC-mediated collagen fibrillogenesis in vitro. FIG. 3A is a graph depicting the effect upon fibrillogenesis of increasing concentration of green fluorescently labeled (FITC) collagen monomers. Using a fluorescence plate reader, fibrillogenesis was quantified as average green fluorescence intensity normalized by cell number (blue fluorescence intensity) for each well. Fibrillogenesis showed a statistically significant increase with collagen concentration (p<0.01 for all concentrations compared to SMC with no added collagen monomers; n=12 samples per collagen monomer concentration). Fibrils did not form in the absence of cells even at the highest concentration used (100 g/mL).

FIG. 3B is a graph showing the time course of fibrillogenesis for midrange collagen monomer concentration (50 g/mL). The average quantified fibrillogenesis (n=12 samples per time point) becomes statistically significant at 6 h and continues to increase throughout the 24 h. (*p<0.05, **p<0.01) Lower panels (fluorescence microscopy): Images show visualization of fibrillogenesis of FITC-labeled collagen monomers, as mediated by cultured mouse aortic SMC (insets: consistent cell seeding indicated by blue-stained cell nuclei) after 6 and 24 h. Fibril formation is first visible at 6 h, with strands of FITC-labeled collagen in a crisscrossed pattern over the cell layer. At 24 h, the fibrillogenesis continues, with collagen fibrils becoming more densely packed and thicker.

On the other hand, when the same FITC-labeled rat-tail-derived collagen molecules, with concentrations increasing from 0-100 g/mL were added to identical numbers of SMC, a gradual and significant increase in collagen assembly was detected, as indicated by increased values of fluorescence, from 0.198±0.012 at 10 g/mL of FITC-collagen to 1.91±0.17 arbitrary units at 100 g/mL of FITC-collagen (p<0.001 for all conditions vs. no collagen).

To investigate the time course of cell-assisted assembly of exogenously added labeled collagen monomers, fibrillogenesis for the intermediate concentration (50 g/mL) was measured at several time points up to 24 h (FIG. 3C-3D). Using the fluorescent microscope, a gradual increase in the number and thickness of collagen fibrils was observed, suggesting overall growth of fibrils with time (FIG. 3D).

Using a fluorescence plate reader, values that were statistically significant after 6 h of incubation (0.064±0.006 vs. 0.042±0.002 arbitrary units for 0 h, p<0.05) were measured. Assembly of collagen continued to increase throughout the 24 h used for the assay (0.467±0.030, p<0.01 compared to 0 h). The fibrillar nature of the assembled collagen was confirmed using transmission electron microscopy (FIGS. 1D-F).

### Example 8

### Effect of Collagen and SMC Source Upon Fibrillogenesis Assay

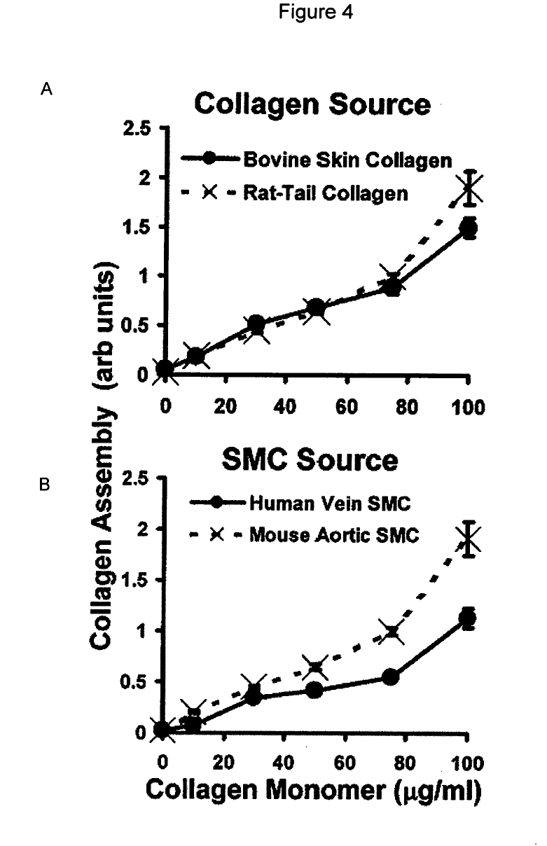

To assess the wider utility of our assay, as various sources of collagen or cells may be used for various applications, SMC-assisted fibrillogenesis of collagen molecules obtained from bovine skin was compared to that of rat tail, the two most commonly used sources, over a similar range of collagen concentrations (FIGS. 4A and B). No statistical significance between mouse SMC-assisted fibrillogenesis of collagen from these two sources was found at any concentration tested.

FIG. 4 shows the effects of collagen or SMC source on cell-assisted collagen fibrillogenesis. FIG. 4A is a graph showing the comparison of fibrillogenesis of collagen from rat-tail or bovine-skin collagen by mouse aortic SMC indicated no statistical differences between these two major experimental sources of collagen. FIG. 4B is a graph showing the comparison between fibrillogenesis of rat-tail collagen mediated by human saphenous vein SMC or by mouse aortic SMC revealed the same concentration dependence although the actual values were different, as expected for cells coming from two different sources, indicating the potential utility of this assay across cell sources and its sensitivity.

The overall trend obtained with SMC from the two different species was similar, with values of fibrillogenesis that almost doubled when the concentration of collagen molecules was increased from 75 g/mL to 100 g/mL for both species (for mouse-derived SMC, from 1.00±0.03 to 1.91±0.16 arbitrary units, p<0.01, compared to human-derived SMC, from 0.55±0.28 to 1.13±0.07 arbitrary units, p<0.01). The assay using human SMC also was sensitive to the concentration of collagen used although, not surprisingly, given the different source of cells, the actual values measured using the human SMC were smaller than those obtained using the mouse SMC with the same concentrations of collagen.

### Example 9

### Treatment Effects on SMC-Mediated Collagen Fibrillogenesis

The current understanding of cell participation to formation of fibrils through assembly of collagen molecules suggests that it is initiated at the cell surface, potentially through the use of integrin receptors, with fibril growth facilitated through the continuous addition of collagen molecules to the growing fibril (FIGS. 3A-D). Thus, initial fibril formation should be influenced by changes in the cell cytoskeleton.

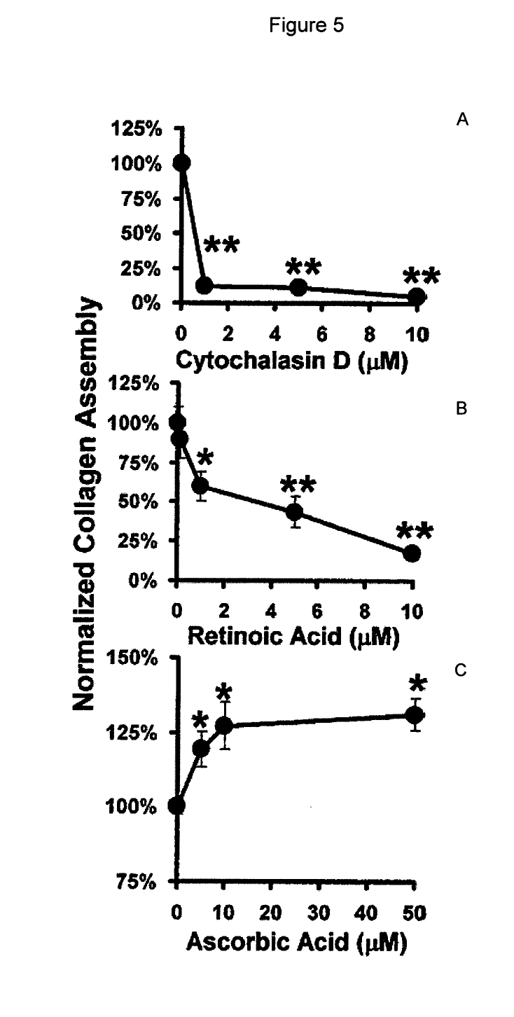

The effect of disrupting the SMC actin cytoskeleton using cytochalasin D upon fibrillogenesis was investigated (FIG. 5). The assay proved to be extremely sensitive, with a significant reduction in fibrillogenesis (12.1±1.0% fibrillogenesis of no treatment control, p<0.001) already detectable at the smallest concentration of cytochalasin D used (1 M). The results support the notion that SMC-mediated collagen fibrillogenesis is actin dependent, and the assay is valuable for highlighting such cell-dependent effects upon collagen assembly.

Exemplary assays can be used to reveal the effect of SMC treatments upon collagen fibrillogenesis. Fibrillogenesis is quantified as fibril accumulation (green fluorescence) normalized by cell density (blue fluorescence). FIG. 5A illustrates that disruption of the actin cytoskeleton, using increasing amounts of cytochalasin D, impairs the ability of SMC to facilitate fibrillogenesis. FIG. 5B illustrates that retinoic acid, a cell-differentiating agent also impairs SMC-assisted collagen fibrillogenesis. Treatment with retinoic acid significantly decreased the ability of SMC to mediate collagen fibrillogenesis starting at 1 M of retinoic acid (59.5±9.4% fibrillogenesis of no treatment control, p<0.001). The effect continued to be augmented by increased concentrations of retinoic acid.

FIG. 5C indicates that the assay is able to detect the contribution of increased production of endogenous collagen by SMC treated with ascorbic acid. De novo synthesized collagen likely is incorporated in the fibrils, thus resulting in a moderate, yet statistically significant, enhancement of collagen fibrils. (*p<0.05, **p<0.01).

To investigate whether or not stabilization of assembled collagen is necessary for the assay, the effects of homocysteine thiolactone were investigated. Homocystein thiolactone is an inhibitor of lysyl oxidase,[13] an enzyme involved in the crosslinking of collagen. However, up to concentrations twice the IC₅₀ value, no effects in SMC incubated with homocysteine thiolactone were detected, (data not shown). This result confirms that lysyl oxidase is not necessary for assembly of the exogenous collagen.

1. A method for identifying modulators of cell-mediated extracellular polymer formation comprising:
(a) incubating a living cell with a plurality of labeled monomers in the presence of a test compound under conditions which promote cell-mediated extracellular polymer formation;
(b) detecting labeled monomers incorporated into a cell-mediated extracellular polymer formed by the cell in step (a); and
(c) comparing detectable cell-mediated polymers formed in step (a) with detectable cell-mediated polymers formed by a cell in the absence of the test compound, wherein a difference in detectable cell-mediated polymers formed in step (a) and by the cell in the absence of the test compound indicates that the test compound modulates cell-mediated extracellular polymer formation.

2. The method of claim 1, wherein the cell is a mammalian cell.

3. The method of claim 2, wherein the cell is a primary culture cell, transfected cell, or immortalized cell.

4. The method of claim 1, wherein the cell is a smooth muscle cell, stem cell, pluripotent cell, undifferentiated cell, or a differentiated cell.

5. The method of claim 1, wherein the cell-mediated polymer is a protein.

6. The method of claim 5, wherein the cell-mediated polymer is collagen.

7. The method of claim 1, wherein the monomer is selected from the group consisting of an amino acid, a nucleic acid, a sugar, or a lipid.

8. The method of claim 1, wherein the label is selected from the group consisting of a chromophore, fluorophore, radioisotope, ¹⁴C, tritium, spin label, nanoparticle, metal particle, biotin, or an enzyme.

9. The method of claim 1, wherein the labeled cell-mediated polymers are detected in real-time.

10. The method of claim 9, wherein the labeled cell-mediated polymers are detected using a fluorescence optical imaging device.

11. The method of claim 1, wherein the method is automated for high-throughput screening.

12. A method for identifying modulators of cell-mediated extracellular polymer formation comprising:
(a) incubating a living cell in a three-dimensional scaffold with a plurality of labeled monomers in the presence of a test compound under conditions which promote cell-mediated extracellular polymer formation;
(b) detecting labeled monomers incorporated into a cell-mediated extracellular polymer formed by the cell in step (a); and
(c) comparing detectable cell-mediated polymers formed in step (a) with detectable cell-mediated polymers formed by a cell in the absence of the test compound, wherein a difference in detectable cell-mediated polymers formed in step (a) and by the cell in the absence of the test compound indicates that the test compound modulates cell-mediated extracellular polymer formation.

13. The method of claim 12, wherein the cell is a mammalian cell.

14. The method of claim 13, wherein the cell is a primary culture cell, transfected cell, or immortalized cell.

15. The method of claim 12, wherein the cell is a smooth muscle cell, stem cell, pluripotent cell, undifferentiated cell, or a differentiated cell.

16. The method of claim 12, wherein the cell-mediated polymer is a protein.

17. The method of claim 16, wherein the cell-mediated polymer is collagen.

18. The method of claim 12, wherein the monomer is selected from the group consisting of an amino acid, a nucleic acid, a sugar, or a lipid.

19. The method of claim 12, wherein the label is selected from the group consisting of a chromophore, fluorophore, radioisotope, ¹⁴C, tritium, spin label, nanoparticle, metal particle, biotin, or an enzyme.

20. The method of claim 12, wherein the labeled cell-mediated polymers are detected in real-time.

21. The method of claim 20, wherein the labeled cell-mediated polymers are detected using a fluorescence optical imaging device.

22. The method of claim 12, wherein the method is automated for high-throughput screening.

23. A method for identifying modulators of cell-mediated extracellular supramolecular polymer formation comprising:
(a) incubating a living cell with a plurality of labeled polymeric subunits in the presence of a test compound under conditions which promote cell-mediated extracellular supramolecular polymer formation;
(b) detecting labeled subunits incorporated into a cell-mediated extracellular polymer formed by the cell in step (a); and
(c) comparing detectable cell-mediated polymers formed in step (a) with detectable cell-mediated polymers formed by a cell in the absence of the test compound, wherein a difference in detectable cell-mediated polymers formed in step (a) and by the cell in the absence of the test compound indicates that the test compound modulates cell-mediated extracellular polymer formation.

24. The method of claim 23, wherein the living cell is incubated in a three-dimensional scaffold.

25. The method of claim 23, wherein the cell is a mammalian cell.

26. The method of claim 25, wherein the cell is a primary culture cell, transfected cell, or immortalized cell.

27. The method of claim 23, wherein the cell is a smooth muscle cell, stem cell, pluripotent cell, undifferentiated cell, or a differentiated cell.

28. The method of claim 23, wherein the cell-mediated polymer is a protein.

29. The method of claim 28, wherein the cell-mediated polymer is collagen.

30. The method of claim 23, wherein the monomer is selected from the group consisting of an amino acid, a nucleic acid, a sugar, or a lipid.

31. The method of claim 23, wherein the label is selected from the group consisting of a chromophore, fluorophore, radioisotope, ¹⁴C, tritium, spin label, nanoparticle, metal particle, biotin, or an enzyme.

32. The method of claim 23, wherein the labeled cell-mediated polymers are detected in real-time.

33. The method of claim 32, wherein the labeled cell-mediated polymers are detected using a fluorescence optical imaging device.

34. The method of claim 23, wherein the method is automated for high-throughput screening.

35. A method for identifying modulators of cell-mediated extracellular collagen fibrillogenesis comprising:
(a) incubating a living cell with a plurality of labeled collagen monomers in the presence of a test compound under conditions which promote cell-mediated extracellular collagen fibrillogenesis;
(b) detecting labeled monomers incorporated into a cell-mediated extracellular collagen fibril formed by the cell in step (a); and
(c) comparing detectable cell-mediated collagen fibers formed in step (a) with detectable cell-mediated collagen fibrils formed by a cell in the absence of the test compound, wherein a difference in detectable cell-mediated collagen fibrils formed in step (a) and by the cell in the absence of the test compound indicates that the test compound modulates cell-mediated extracellular collagen fibrillogenesis.

36. The method of claim 35, wherein the living cell is incubated in a three-dimensional scaffold.

37. The method of claim 35, wherein the cell is a mammalian cell.

38. The method of claim 37, wherein the cell is a primary culture cell, transfected cell, or immortalized cell.

39. The method of claim 35, wherein the cell is a smooth muscle cell, stem cell, pluripotent cell, undifferentiated cell, or a differentiated cell.

40. The method of claim 35, wherein the label is selected from the group consisting of a chromophore, fluorophore, radioisotope, ¹⁴C, tritium, spin label, nanoparticle, metal particle, biotin, or an enzyme.

41. The method of claim 35, wherein the labeled cell-mediated collagen fibrils are detected in real-time.

42. The method of claim 35, wherein the labeled cell-mediated fibrils are detected using a fluorescence optical imaging device.

43. The method of claim 35, wherein the method is automated for high-throughput screening.

44. A kit for detecting or quantifying cell-mediated polymer assembly comprising:
(a) a cell culture plate optionally comprising a scaffold; and
(b) labeled monomers of a predetermined cell-mediated polymer, unlabeled monomers, labeling reagents, or a combination thereof.
